(19) United States
(12) Patent Application Publication
(10) Pub. No.: US 2016/0193276 A1
(43) Pub. Date: Jul. 7, 2016

(54) TRANSMUCOSAL DELIVERY OF GLATIRAMER ACETATE

(71) Applicants: Ursula GEISTER, Blaubeuren (DE); Stephan SCHWEIZER, Ehigen (DE); Martina BUERGER, Ulm (DE); Ralph STEFAN, Ebenweiler (DE); Gerald HUBER, Pluderhausen (DE); Tanja PRIES, Munich (DE)

(72) Inventors: Ursula GEISTER, Blaubeuren (DE); Stephan SCHWEIZER, Ehingen (DE); Martina BUERGER, Ulm (DE); Ralph STEFAN, Ebenweiler (DE); Gerald HUBER, Plüderhausen (DE); Tanja PRIES, Munich (DE)

(73) Assignee: Teva Pharmaceutical Industries, Ltd., Petach-Tikva (IL)

(21) Appl. No.: 14/653,022

(22) PCT Filed: Dec. 20, 2013
(86) PCT No.: PCT/US13/77034

§ 371 Date: Jun. 17, 2015

Related U.S. Application Data
(60) Provisional application No. 61/745,226, filed on Dec. 21, 2012.

(51) Int. Cl. A61K 38/03; A61K 9/20; A61K 9/00

(52) U.S. Cl. CPC A61K 38/03; A61K 9/006; A61K 9/2054; A61K 9/2072; A61K 9/2027

(57) ABSTRACT

The present invention provides an oral tablet comprising glatiramer acetate in an amount from about 10 percent to about 60 percent by weight and one or more gel forming agents in a total amount up to about 90 percent by weight. The present invention also provides a method of delivering glatiramer acetate across a buccal membrane comprising orally administering an oral tablet of any one of the embodiments.

This application claims priority of U.S. Provisional Application No. 61/745,226, filed Dec. 21, 2012, the entire content of which is hereby incorporated by reference herein.

Throughout this application various publications are referenced. The disclosures of these publications in their entireties are hereby incorporated by reference into this application in order to more fully describe the state of the art to which this invention pertains.

### BACKGROUND OF THE INVENTION

Multiple Sclerosis

Multiple Sclerosis (MS) is a chronic, debilitating disease of the central nervous system (CNS). MS has also been classified as an autoimmune disease. MS disease activity can be monitored by magnetic resonance imaging (MRI) of the brain, accumulation of disability, as well as rate and severity of relapses.

There are five main forms of multiple sclerosis:

1) Benign Multiple Sclerosis:

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis.

2) Relapsing-Remitting Multiple Sclerosis (RRMS):

Patients suffering from RRMS experience sporadic exacerbations or relapses, as well as periods of remission. Lesions and evidence of axonal loss may or may not be visible on MRI for patients with RRMS.

3) Secondary Progressive Multiple Sclerosis (SPMS):

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.

4) Primary Progressive Multiple Sclerosis (PPMS);

PPMS is characterized by a steady progression of increasing neurological deficits without distinct attacks or remissions. Cerebral lesions, diffuse spinal cord damage and evidence of axonal loss are evident on the MRI of patients with PPMS.

5) Progressive-Relapsing Multiple Sclerosis (PRMS):

PRMS has periods of acute exacerbations while proceeding along a course of increasing neurological deficits without remissions. Lesions are evident on MRI of patients suffering from PRMS (Multiple sclerosis: its diagnosis, symptoms, types and stages, 2003, albany.net/.about.tjc/multiple-sclerosis.html; What are the Types of Multiple Sclerosis?, 2005, <imaginis.com/multiple-sclerosis/types-of-ms.asp? mode=1>).

Chronic progressive multiple sclerosis is a term used to collectively refer to SPMS, PPMS, and PRMS (Types of Multiple Sclerosis (MS), 2005, <themcfox.com/multiple-sclerosis/types-of-ms/types-of-multi-ple-sclerosis.htm>). The relapsing forms of multiple sclerosis are SPMS with superimposed relapses, RRMS and PRMS.

A clinically isolated syndrome (CIS) is a single monosymptomatic attack compatible with MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process (Frohman et al., The utility of MRI in suspected MS: report of the Therapeutics and Technology Assessment Subcommittee of the American Academy of Neurology, Neurology 61(5):602-11 (2003)).

Multiple sclerosis may present with optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

Glatiramer Acetate

Glatiramer acetate (GA), a mixture of polypeptides which do not all have the same amino acid sequence, is marketed under the tradename Copaxone®. GA comprises the acetate salts of polypeptides containing L-glutamic acid, L-alanine, L-tyrosine and L-lysine at average molar fractions of 0.141, 0.427, 0.095 and 0.338, respectively. The average molecular weight of Copaxone® is between 5,000 and 9,000 daltons. (“Copaxone”, Physician's Desk Reference, (2005), Medical Economics Co., Inc., (Montvale, N.J.), 3115.) Chemically, glatiramer acetate is designated L-glutamic acid polymer with L-alanine, L-lysine, L-tyrosine, acetate (salt).

Its structural formula is:

(Glu,Ala,Lys,Tyr).xCH₃COOH(C₅H₉NO₄.C₃H₇NO₂.C₆H₁₄N₂O₂.C₉H₁₁NO₃).xC₂H₄O₂

CAS-147245-92-9

Copaxone® (“Copaxone”, Full Prescribing Information, (February, 2009), FDA Marketing Label) (20 mg glatiramer acetate daily injection) is an approved therapy for patients with relapsing remitting multiple sclerosis (RRMS), including patients who have experienced a first clinical episode and have MRI features consistent with multiple sclerosis.

GA has also been disclosed for use in the treatment of other autoimmune diseases (U.S. Patent Publication No. 2002/0055466 A1 (R. Aharoni et al.), inflammatory non-autoimmune diseases (U.S. Patent Publication No. 2005/0014694 A1 (V. Wee Yong et al.); and U.S. Patent Application No. 2002/0077278 A1, published Jun. 20, 2002 (Young et al.)) and other diseases (U.S. Patent Publication Nos. 2003/0004099 A1 and 2002/0037848 A1 (Eisenbach-Schwartz, et al.); U.S. Pat. No. 6,514,938 B1, issued Feb. 4, 2003 (Gad et al.); PCT International Publication No. WO 01/60392, published Aug. 23, 2001 (Gilbert et al.); PCT International Publication No. WO 00/27417, published May 19, 2000 (Aharoni et al.); and PCT International Publication No. WO 01/97846, published Dec. 27, 2001 (Moses et al.).

The 20 mg/day subcutaneous (s.c.) dose has been shown to reduce the total number of enhancing lesions in MS patients as measured by MRI (G. Comi et al., European/Canadian Multicenter, Double-Blind, Randomized, Placebo-Controlled Study of the Effects of Glatiramer Acetere on Magnetic Resonance Imaging-Measured Disease Activity and Burden in Patients with Relapsing Multiple Sclerosis, Ann. Neurol. 49:290-297 (2001)).

Safety data accumulated for GA in clinical trials shows that the drug product is safe and well tolerated. However, reactions including Immediate Post-Injection Reaction (IPIR) consisting of one or more of the following symptoms: vasodilatation, chest pain, dyspnoea, palpitations or tachycardia was reported for 31% of the GA patients vs. 13% on placebo. Additional adverse reactions reported by patients treated with GA 20 mg with at least 2% higher incidence than with placebo were pain, nausea, anxiety, rash, back pain, chills, face edema, local reaction, lymphadenopathy, vomiting, weight increase, tremor, skin disorder, eye disorder, vaginal candidiasis and injection site atrophy.

In all clinical trials, injection-site reactions were seen to be the most frequent adverse reactions and were reported by the majority of patients receiving GA. In controlled studies, the proportion of patients reporting these reactions, at least once, was higher following treatment with GA (70%) than placebo injections (37%). The most commonly reported injection-site reactions, which were more frequently reported in GA vs. placebo-treated patients, were erythema, pain, mass, pruritus, edema, inflammation and hypersensitivity.

In addition to the observed adverse events, administration by injection can be burdensome which can lead to poor patient compliance or suspension of therapy. Accordingly, there exists a need to develop alternative routes of glatiramer acetate delivery in which the glatiramer acetate is effective in treating a symptom of a form of multiple sclerosis.

Alternatives to Glatiramer Acetate Injection

Glatiramer acetate administration through ingestion or inhalation has been disclosed (U.S. Pat. No. 6,214,791); and compositions for oral, nasal and pulmonary administration also have been disclosed (U.S. Patent Application Publication No. 2001/0055568 A1).

Studies in mice showed that orally administered glatiramer acetate inhibited the induction of experimental autoimmune encephalomyelitis (EAE) in rats and mice and suggested that oral administration of glatiramer acetate may modulate multiple sclerosis as well (Teitelbaum et al., Immunomodulation of experimental autoimmune encephalomyelitis by oral administration of copolymer 1, Immunology 96:3842-3847 (1999)). However, alternative routes of administration have yet to be demonstrated to be effective in the treatment of multiple sclerosis. For example, glatiramer acetate administered orally did not affect relapse rate or other clinical MRI parameters of disease activity in a recent clinical trial (Filippi et al, Effects of oral glatiramer acetate on clinical and MRI-monitored disease activity in patients with relapsing multiple sclerosis: a multicentre, double-blind, randomised, placebo-controlled study, Lancet Neurol. 5(3):213-220 (2006)).

Buccal administration avoids hepatic metabolism and gastrointestinal degradation which can hinder effectiveness of orally administered drugs and provides an attractive alternative to oral administration. However, the buccal mucosa is not an absorptive organ and permeation of the drug to be administered is problematic. Other problems to be overcome include drug stability and formulation palatability.

Advantages of Mucoadhesive Buccal Drug Delivery System

Drugs administered via oral mucosa offers several advantages
Ease of administration.
Termination of therapy is easy.
Permits localization of drug to the oral cavity for a prolonged period of time.
Can be administered to unconscious patients.
Offers an excellent route, for the systemic delivery of drugs with high first pass metabolism, thereby offering a greater bioavailability.
A significant reduction in dose can be achieved there by reducing dose related side effects.
Drugs which are unstable in the acidic environment are destroyed by enzymatic or alkaline environment of intestine can be administered by this route.
Drugs which show poor bioavailability via the oral route can be administered conveniently.
It offers a passive system of drug absorption and does not require any activation.
The presence of saliva ensures relatively large amount of water for drug dissolution unlike in case of rectal and transdermal routes.
Systemic absorption is rapid.
This route provides an alternative for the administration of various hormones, narcotic analgesic, steroids, enzymes, cardiovascular agents etc.
The buccal mucosa is highly perfused with blood vessels and offers a greater permeability than the skin.

### SUMMARY OF THE INVENTION

The present invention provides an oral tablet comprising glatiramer acetate in an amount from about 10 percent to about 60 percent by weight and one or more gel forming agents in a total amount up to about 90 percent by weight.

The present invention provides an oral tablet comprising glatiramer acetate in an amount from about 10 percent to about 40 percent by weight and one or more gel forming agents in a total amount up to about 90 percent by weight.

The present invention also provides an oral tablet comprising about 40 mg glatiramer acetate, about 80 mg carbomer, about 25 mg hydroxypropylcellulose, about 30.5 mg mannitol, about 1.5 mg colloidal silicon dioxide and about 3 mg sodium stearyl fumerate.

The present invention also provides an oral tablet comprising about 20 mg glatiramer acetate, about 50 mg carbomer, about 15 mg hydroxypropylcellulose, about 12 mg mannitol, about 1 mg colloidal silicon dioxide and about 2 mg sodium stearyl fumerate.

The present invention also provides an oral tablet comprising about 40 mg glatiramer acetate, about 80 mg chitosan, about 45 mg hydroxypropylcellulose, about 10.5 mg mannitol, about 1.5 mg colloidal silicon dioxide and about 3 mg sodium stearyl fumerate.

The present invention also provides an oral tablet comprising about 20 mg glatiramer acetate, about 45 mg chitosan, about 30 mg hydroxypropylcellulose, about 2 mg mannitol, about 1 mg colloidal silicon dioxide and about 2 mg sodium stearyl fumerate.

The present invention also provides an oral tablet comprising about 40 mg glatiramer acetate, about 80 mg thiolated chitosan, about 45 mg hydroxypropylcellulose, about 10.5 mg mannitol, about 1.5 mg colloidal silicon dioxide and about 3 mg sodium stearyl fumerate.

The present invention also provides an oral tablet comprising about 20 mg glatiramer acetate, about 45 mg thiolated chitosan, about 30 mg hydroxypropylcellulose, about 2 mg mannitol, about 1 mg colloidal silicon dioxide and about 2 mg sodium stearyl fumerate.

The present invention also provides an oral tablet comprising about 40 mg glatiramer acetate, about 80 mg thiolated carbomer, about 25 mg hydroxypropylcellulose, about 30.5 mg mannitol, about 1.5 mg colloidal silicon dioxide and about 3 mg sodium stearyl fumerate.

The present invention also provides an oral tablet comprising about 20 mg glatiramer acetate, about 50 mg thiolated carbomer, about 15 mg hydroxypropylcellulose, about 12 mg mannitol, about 1 mg colloidal silicon dioxide and about 2 mg sodium stearyl fumerate.

The present invention also provides an oral tablet comprising about 50 mg glatiramer acetate, about 40 mg carbomer, about 10 mg hydroxypropyl cellulose, about 47 mg mannitol, about 1 mg fumed silica and about 2 mg sodium stearyl fumerate.

The present invention also provides a method of delivering glatiramer acetate across a buccal membrane comprising orally administering an oral tablet of any one of the embodiments.

The present invention also provides a process of making a pharmaceutical composition containing glatiramer acetate, comprising the steps of:
a) mixing the glatiramer acetate with one of more excipients under dry conditions; and
b) forming the pharmaceutical composition.

### BRIEF DESCRIPTION OF THE DRAWINGS

FIG. 1 shows a buccal tablet prepared according to Example 7 which has been placed on glass in a small amount of water for two hours to simulate conditions in the buccal pouch.

FIG. 2 shows the average permeation of the different formulations. Data series are presented as follows: tablets (diamond markers), glatiramer acetate solution without pre-incubated tissue (square markers, solid line) and glatiramer acetate solution with DMSO pre-incubated tissue (square markers, dotted line).

### DETAILED DESCRIPTION OF THE INVENTION

### Terms

As used in this application, except as otherwise expressly provided herein, each of the following terms shall have the meaning set forth below.

As used herein, an “amount” or “dose” of an agent measured in milligrams refers to the milligrams of agent present in a drug product, regardless of the form of the drug product.

Administration of different amounts of glatiramer acetate using oral tablet of the present invention can be accomplished by using one, two, three, four or five oral tablets at the same time or consecutively or by using a portion of an oral tablet. For example ½ of an oral tablet can be obtained by cutting an oral tablet once and ¼ of an oral tablet can be obtained by cutting an oral tablet twice.

Administration of an amount from about 5 to about 200 mg of glatiramer acetate can be achieved using the oral tablets of the present invention. For Example, administration of 5 mg glatiramer acetate can be accomplished by using ¼ of an oral tablet containing 20 mg glatiramer acetate and administration of 10 mg glatiramer acetate can be accomplished by using of an oral tablet containing 20 mg glatiramer acetate. Likewise, administration of 20, 40, 60, 80 or 100 mg glatiramer acetate can be accomplished by using 1, 2, 3, 4 or 5 oral tablets containing 20 mg glatiramer acetate, respectively. Similarly, administration of 25 mg glatiramer acetate can be accomplished by using of an oral tablet containing 50 mg glatiramer acetate and administration of 50, 100, 150 or 200 mg glatiramer acetate can be accomplished using 1, 2, 3 or 4 oral tablets containing 50 mg glatiramer acetate acetate, respectively. Similarly, administration of 100 mg glatiramer acetate can be accomplished, for example, by using a single oral tablet containing 100 mg glatiramer acetate, or by using 2 oral tablets containing 50 mg glatiramer acetate, etc.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “gel forming agents” are agents which form a matrix which allows for controlled release of an active ingredient. Gel forming agents include, but are not limited to, carbomer, carbomer (sodium salt), hydroxypropylcellulose, chitosan, thiolated chitosan, thiolated carbomer, ethylcellulose, gelatine, hydroxyethylcellulose, methylcellulose, carboxymethylcellulose, gummi arabicum, xanthan gum and carrageen. Gel forming agents are commercially available under numerous trade names. For example, hydroxypropyl cellulose is available as Klucel® or Klucel® HF.

As used herein, “glidants” are agents which improve flow in a powdered mixture. Glidants include, but are not limited to, colloidal silicon dioxide colloidal silicon dioxide, a fumed silica, a hydrophobic fumed silica such as Aerosil® or Aerosil® 200 a magnesium aluminometasilicate such as Neusilin® and magnesium stearate.

As used herein, “lubricants” include, but are not limited to a stearate, a stearyl fumerate such as sodium stearyl fumerate or Pruv®, talcum powder or fatty acid, glycerol dibehenate, more preferably, hexanedioic acid or an earth alkali metal stearate, such as magnesium stearate.

As used herein, an “oral tablet” is a tablet designed to be administered in the oral cavity, it includes tablets designed to be administered between the cheek and gum and tablets designed to be administered sublingually. In one or more embodiments of the present invention the oral tablet is a mucoadhesive oral tablet.

Carbomers are synthetic high-molecular-weight polymers of acrylic acid that are crosslinked with either allyl sucrose or allyl ethers of pentaerythritol. They contain between 52% and 68% of carboxylic acid (COOH) groups calculated on the dry basis. The BP 2009 and PhEur 6.4 have a single monograph describing carbomer; the USP32-NF27 contains several monographs describing individual carbomer grades that vary in aqueous viscosity, polymer type, and polymerization solvent. The molecular weight of carbomer is theoretically estimated at 7×10⁵ to 4×10⁹. Carbomers are commercially available under numerous trade names. For example, Carbopol® or Carbopol® 974 P.

Relapsing Form of Multiple Sclerosis:

The term relapsing MS includes:
1) patients with RRMS;
2) patients with SPMS and superimposed relapses; and
3) patients with CIS who show lesion dissemination on subsequent MRI scans according to McDonald's criteria.

As used herein, relapsing forms of multiple sclerosis include: Relapsing-remitting multiple sclerosis (RRMS), characterized by unpredictable acute episodes of neurological dysfunction (relapses), followed by variable recovery and periods of clinical stability;

Secondary Progressive MS (SPMS), wherein patients having RRMS develop sustained deterioration with or without relapses superimposed; and

Primary progressive-relapsing multiple sclerosis (PPRMS) or progressive-relapsing multiple sclerosis (PRMS), an uncommon form wherein patients developing a progressive deterioration from the beginning can also develop relapses later on.

### EMBODIMENTS OF THE INVENTION

The present invention provides an oral tablet comprising glatiramer acetate in an amount from about 10 percent to about 60 percent by weight and one or more gel forming agents in a total amount up to about 90 percent by weight.

The present invention provides an oral tablet comprising glatiramer acetate in an amount from about 10 percent to about 40 percent by weight and one or more gel forming agents in a total amount up to about 90 percent by weight.

In one or more embodiments of the present invention, the glatiramer acetate is present in an amount from about 15 percent to about 30 percent by weight.

In one or more embodiments of the present invention, the glatiramer acetate is present in an amount from about 17 percent to about 25 percent by weight.

In one or more embodiments of the present invention, the glatiramer acetate is present in an amount from about 20 percent to about 23 percent by weight.

In one or more embodiments of the present invention, the glatiramer acetate is present in an amount from about 15 percent to about 50 percent by weight.

In one or more embodiments of the present invention, the glatiramer acetate is present in an amount from about 25 percent to about 40 percent by weight.

In one or more embodiments of the present invention, the glatiramer acetate is present in an amount from about 30 percent to about 35 percent by weight.

In one or more embodiments of the present invention, the glatiramer acetate is present in an amount of about 33 percent by weight.

In one or more embodiments of the present invention, the glatiramer acetate is present in an amount of about 20 percent by weight.

In one or more embodiments of the present invention, the glatiramer acetate is present in an amount of about 22 percent by weight.

In one or more embodiments of the present invention, the one or more gel forming agents are present in a total amount from about 20 percent to about 90 percent by weight.

In one or more embodiments of the present invention, the one or more gel forming agents are present in a total amount from about 40 percent to about 90 percent by weight.

In one or more embodiments of the present invention, the one or more gel forming agents are present in a total amount from about 50 percent to about 80 percent by weight.

In one or more embodiments of the present invention, the one or more gel forming agents are present in a total amount from about 55 percent to about 75 percent by weight.

In one or more embodiments of the present invention, the one or more gel forming agents are present in a total amount from about 20 percent to about 40 percent by weight.

In one or more embodiments of the present invention, the one or more gel forming agents are present in a total amount from about 30 percent to about 35 percent by weight.

In one or more embodiments of the present invention, the one or more gel forming agents are present in a total amount of about 33 percent by weight.

In one or more embodiments of the present invention, the oral tablet further comprises a filler present in an amount up to about 80 percent by weight.

In one or more embodiments of the present invention, the filler is present in an amount up to about 60 percent by weight.

In one or more embodiments of the present invention, the filler is present in an amount up to about 50 percent by weight.

In one or more embodiments of the present invention, the filler is present in an amount up to about 40 percent by weight.

In one or more embodiments of the present invention, the filler is present in an amount up to about 20 percent by weight.

In one or more embodiments of the present invention, the filler is present in an amount from about 2 percent to about 17 percent by weight.

In one or more embodiments of the present invention, the filler is present in an amount from about 15 percent to about 50 percent by weight.

In one or more embodiments of the present invention, the filler is present in an amount from about 25 percent to about 40 percent by weight.

In one or more embodiments of the present invention, the filler is present in an amount from about 30 percent to about 35 percent by weight.

In one or more embodiments of the present invention, the filler is present in an amount of about 31 percent by weight.

In one or more embodiments of the present invention, the oral tablet further comprises a glidant present in an amount up to about 3 percent by weight.

In one or more embodiments of the present invention, the glidant is present in an amount from about 0.5 percent to about 1.5 percent by weight.

In one or more embodiments of the present invention, the glidant is present in an amount from about 0.75 percent to about 1.25 percent by weight.

In one or more embodiments of the present invention, the glidant is present in an amount from about 0.3 percent to about 1.0 percent by weight.

In one or more embodiments of the present invention, the glidant is present in an amount from about 0.6 percent to about 0.8 percent by weight.

In one or more embodiments of the present invention, the glidant is present in an amount of about 0.7 percent by weight.

In one or more embodiments of the present invention, the oral tablet further comprises a lubricant present in an amount up to about 10 percent by weight.

In one or more embodiments of the present invention, the lubricant present in an amount up to about 5 percent by weight.

In one or more embodiments of the present invention, the lubricant is present in an amount from about 1 percent to about 3 percent by weight.

In one or more embodiments of the present invention, the lubricant is present in an amount from about 1 percent to about 1.5 percent by weight.

In one or more embodiments of the present invention, the lubricant is present in an amount of about 1.3 percent by weight.

In one or more embodiments of the present invention, the lubricant is present in an amount from about 1.5 percent to about 2 percent by weight.

In one or more embodiments of the present invention, the one or more gel forming agents are selected from the group consisting of carbomer, carbomer (sodium salt), hydroxypropylcellulose, chitosan, thiolated chitosan, thiolated carbomer, ethylcellulose, gelatine, hydroxyethylcellulose, methylcellulose, carboxymethylcellulose, sodium carboxymethlcellulose, gummi arabicum, xanthan gum, carboxymethyl cellulose and carrageen.

In one or more embodiments of the present invention, the one or more gel forming agents are selected from the group consisting of carbomer, hydroxypropylcellulose, chitosan, thiolated chitosan, thiolated carbomer, ethylcellulose, gelatine, hydroxyethylcellulose, methylcellulose, carboxymethylcellulose, gummi arabicum, xanthan gum, carboxymethyl cellulose and carrageen.

In one or more embodiments of the present invention, the filler is selected from the group consisting of mannitol, lactose, saccharose, sucrose, dextrose, isomalt, sorbitol, calcium phosphate, calcium carbonate, calcium silicate, magnesium carbonate, magnesium oxide, glucopyranosyl mannitol and calcium sulfate.

In one or more embodiments of the present invention, the filler is mannitol. In one or more embodiments, the mannitol is Parteck® M or Parteck® M 200.

In one or more embodiments of the present invention, the glidant is selected from the group consisting of silicon dioxide, colloidal silicon dioxide, a fumed silica, a hydrophobic fumed silica, a magnesium aluminometasilicate such as Neusilin® and magnesium stearate.

In one or more embodiments of the present invention, the glidant is selected from the group consisting of silicon dioxide, colloidal silicon dioxide, Neusilin and magnesium stearate.

In one or more embodiments of the present invention, the lubricant is selected from the group consisting of sodium stearyl fumerate, a stearate, talcum powder, a fatty acid, glycerol dibehenate, hexadecanoic acid, polyethylene glycol (PEG) and magnesium stearate.

In one or more embodiments of the present invention, the one or more gel forming agents comprises carbomer, wherein the carbomer is present in an amount from about 10 percent to about 60 percent by weight.

In one or more embodiments of the present invention, the one or more gel forming agents comprises carbomer, wherein the carbomer is present in an amount from about 20 percent to about 60 percent by weight.

In one or more embodiments of the present invention, the carbomer is present in an amount from about 40 percent to about 55 percent by weight.

In one or more embodiments of the present invention, the carbomer is present in an amount from about 44 percent to about 50 percent by weight.

In one or more embodiments of the present invention, the carbomer is present in an amount of about 44 percent by weight.

In one or more embodiments of the present invention, the carbomer is present in an amount of about 50 percent by weight.

In one or more embodiments of the present invention, the carbomer is present in an amount from about 20 percent to about 35 percent by weight.

In one or more embodiments of the present invention, the carbomer is present in an amount from about 25 percent to about 30 percent by weight.

In one or more embodiments of the present invention, the carbomer is present in an amount of about 27 percent by weight.

In one or embodiments of the present invention, the carbomer is Carbopol 974P.

In one or more embodiments of the present invention, the one or more gel forming agents comprises hydroxypropylcellulose, wherein the hydroxypropylcellulose is present in an amount from about 1 percent to about 40 percent by weight.

In one or more embodiments of the present invention, the hydroxypropylcellulose is present in an amount from about 13 percent to about 30 percent by weight.

In one or more embodiments of the present invention, the hydroxypropylcellulose is present in an amount from about 13 percent to about 15 percent by weight.

In one or more embodiments of the present invention, the hydroxypropylcellulose is present in an amount of about 14 percent by weight.

In one or more embodiments of the present invention, the hydroxypropylcellulose is present in an amount of about 15 percent by weight.

In one or more embodiments of the present invention, the hydroxypropylcellulose is present in an amount from about 25 percent to about 30 percent by weight.

In one or more embodiments of the present invention, the hydroxypropylcellulose is present in an amount of about 25 percent by weight.

In one or more embodiments of the present invention, the hydroxypropylcellulose is present in an amount of about 30 percent by weight.

In one or more embodiments of the present invention, the hydroxypropylcellulose is present in an amount from about 2 percent to about 10 percent by weight.

In one or more embodiments of the present invention, the hydroxypropylcellulose is present in an amount from about 6 percent to about 8 percent by weight.

In one or more embodiments of the present invention, the hydroxypropylcellulose is present in an amount of about 7 percent by weight.

In one or more embodiments of the present invention, the one or more gel forming agents comprises chitosan, wherein the chitosan is present in an amount from about 20 percent to about 60 percent by weight.

In one or more embodiments of the present invention, the chitosan is present in an amount from about 40 percent to about 50 percent by weight.

In one or more embodiments of the present invention, the chitosan is present in an amount from about 44 percent to about 45 percent by weight.

In one or more embodiments of the present invention, the chitosan is present in an amount of about 44 percent by weight.

In one or more embodiments of the present invention, the chitosan is present in an amount of about 45 percent by weight.

In one or more embodiments of the present invention, the one or more gel forming agents comprises thiolated chitosan, wherein the thiolated chitosan is present in an amount from about 20 percent to about 60 percent by weight.

In one or more embodiments of the present invention, the thiolated chitosan is present in an amount from about 40 percent to about 50 percent by weight.

In one or more embodiments of the present invention, the thiolated chitosan is present in an amount from about 44 percent to about 45 percent by weight.

In one or more embodiments of the present invention, the thiolated chitosan is present in an amount of about 44 percent by weight.

In one or more embodiments of the present invention, the thiolated chitosan is present in an amount of about 45 percent by weight.

In one or more embodiments of the present invention, the one or more gel forming agents comprises thiolated carbomer, wherein the thiolated carbomer is present in an amount from about 20 percent to about 60 percent by weight.

In one or more embodiments of the present invention, the thiolated carbomer is present in an amount from about 40 percent to about 55 percent by weight.

In one or more embodiments of the present invention, the thiolated carbomer is present in an amount from about 44 percent to about 50 percent by weight.

In one or more embodiments of the present invention, the thiolated carbomer is present in an amount of about 44 percent by weight.

In one or more embodiments of the present invention, the thiolated carbomer is present in an amount of about 45 percent by weight.

In one or more embodiments of the present invention, the tablet has a hardness of about 60 Newtons to about 150 Newtons.

In one or more embodiments of the present invention, the tablet has a hardness of about 60 Newtons to about 110 Newtons.

In one or more embodiments of the present invention, the tablet has a hardness of about 70 Newtons to about 120 Newtons.

In one or more embodiments of the present invention, the tablet has a hardness of about 70 Newtons to about 100 Newtons.

In one or more embodiments of the present invention, the tablet has a hardness of about 80 Newtons to about 90 Newtons.

In one or more embodiments of the present invention, the tablet is a round flat tablet.

In one or more embodiments of the present invention, the tablet is a round biconvex tablet.

In one or more embodiments of the present invention, the tablet has a diameter from about 6 mm to about 10 mm.

In one or more embodiments of the present invention, the tablet has a diameter of about 7 mm.

In one or more embodiments of the present invention, the tablet has a diameter of about 9 mm.

In one or more embodiments of the present invention, the tablet has a diameter of about 8 mm.

In one or more embodiments of the present invention, the tablet has a thickness of about 2.6 mm.

In one or more embodiments of the present invention, the tablet contains from about 10 mg glatiramer acetate to about 100 mg glatiramer acetate.

In one or more embodiments of the present invention, the tablet contains from about 20 mg glatiramer acetate to about 40 mg glatiramer acetate.

In one or more embodiments of the present invention, the tablet contains about 20 mg glatiramer acetate.

In one or more embodiments of the present invention, the tablet contains about 40 mg glatiramer acetate.

In one or more embodiments of the present invention, the tablet contains from about 40 mg glatiramer acetate to about 60 mg glatiramer acetate.

In one or more embodiments of the present invention, the tablet contains about 50 mg glatiramer acetate.

The present invention also provides an oral tablet comprising about 40 mg glatiramer acetate, about 80 mg carbomer, about 25 mg hydroxypropylcellulose, about 30.5 mg mannitol, about 1.5 mg colloidal silicon dioxide and about 3 mg sodium stearyl fumerate.

The present invention also provides an oral tablet comprising about 20 mg glatiramer acetate, about 50 mg carbomer, about 15 mg hydroxypropylcellulose, about 12 mg mannitol, about 1 mg colloidal silicon dioxide and about 2 mg sodium stearyl fumerate.

The present invention also provides an oral tablet comprising about 40 mg glatiramer acetate, about 80 mg chitosan, about 45 mg hydroxypropylcellulose, about 10.5 mg mannitol, about 1.5 mg colloidal silicon dioxide and about 3 mg sodium stearyl fumerate.

The present invention also provides an oral tablet comprising about 20 mg glatiramer acetate, about 45 mg chitosan, about 30 mg hydroxypropylcellulose, about 2 mg mannitol, about 1 mg colloidal silicon dioxide and about 2 mg sodium stearyl fumerate.

The present invention also provides an oral tablet comprising about 40 mg glatiramer acetate, about 80 mg thiolated chitosan, about 45 mg hydroxypropylcellulose, about 10.5 mg mannitol, about 1.5 mg colloidal silicon dioxide and about 3 mg sodium stearyl fumerate.

The present invention also provides an oral tablet comprising about 20 mg glatiramer acetate, about 45 mg thiolated chitosan, about 30 mg hydroxypropylcellulose, about 2 mg mannitol, about 1 mg colloidal silicon dioxide and about 2 mg sodium stearyl fumerate.

The present invention also provides an oral tablet comprising about 40 mg glatiramer acetate, about 80 mg thiolated carbomer, about 25 mg hydroxypropylcellulose, about 30.5 mg mannitol, about 1.5 mg colloidal silicon dioxide and about 3 mg sodium stearyl fumerate.

The present invention also provides an oral tablet comprising about 20 mg glatiramer acetate, about 50 mg thiolated carbomer, about 15 mg hydroxypropylcellulose, about 12 mg mannitol, about 1 mg colloidal silicon dioxide and about 2 mg sodium stearyl fumerate.

The present invention also provides an oral tablet comprising about 50 mg glatiramer acetate, about 40 mg carbomer, about 10 mg hydroxypropyl cellulose, about 47 mg mannitol, about 1 mg fumed silica and about 2 mg sodium stearyl fumerate.

The present invention also provides a method of delivering glatiramer acetate across a buccal membrane comprising orally administering an oral tablet of any one of the embodiments.

In one or more embodiments of the present invention, the tablet is placed between the cheek and gum.

In one or more embodiments of the present invention, the tablet is placed sublingually.

In one or more embodiments of the present invention, the tablet is placed in the mouth at bedtime.

The present invention also provides a process of making a pharmaceutical composition containing glatiramer acetate, comprising the steps of:

a) mixing the glatiramer acetate with one of more excipients under dry conditions; and
b) forming the pharmaceutical composition.

In one or more embodiments of the present invention, the pharmaceutical composition is a buccal tablet.

In one or more embodiments of the present invention, step b) is performed by dry granulation.

In one or more embodiments of the present invention, step b) is performed by direct compression.

As used herein, “about” with regard to a stated number encompasses a range of +10 percent to −10 percent of the stated value. By way of example, about 100 mg therefore includes the range 90-110 mg and therefore also includes 90, 91, 92, 93, 94, 95 96, 97, 98, 99, 100, 101, 102, 103, 104, 105, 106, 107, 108, 109 and 110 mg. Accordingly, about 100 mg includes, in an embodiment, 100 mg.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg, 0.5 mg, 0.6 mg etc. up to 5.0 mg.

All combinations of the various elements described herein are within the scope of the invention.

This invention is illustrated in the Experimental Details section which follows. This section is set forth to aid in an understanding of the invention but is not intended to, and should not be construed to; limit in any way the invention as set forth in the claims which follow thereafter.

### EXAMPLES

### First Series of Experiments

### Example 1

### Tablet Formulations

Oral tablets containing 40 mg glatiramer acetate per tablet are prepared with the compositions set for the in Tables 1-4.

TABLE 1

| Ingredient | mg/tablet |
|---|---|
| Glatiramer acetate | 40.00 |
| Carbomer (Carbopol ® 974 P) | 80.00 |
| Hyprolose (hydroxypropylcellulose) | 25.00 |
| Mannitol | 30.50 |
| Colloidal silicon dioxide | 1.50 |
| Sodium stearyl fumarate | 3.00 |
| Tablet weight | 180.00 |

TABLE 2

| Ingredient | mg/tablet |
|---|---|
| Glatiramer acetate | 40.00 |
| Chitosan | 80.00 |
| Hyprolose | 45.00 |
| Mannitol | 10.50 |
| Colloidal silicon dioxide | 1.50 |
| Sodium stearyl fumarate | 3.00 |
| Tablet weight | 180.00 |

TABLE 3

| Ingredient | mg/tablet |
|---|---|
| Glatiramer acetate | 40.00 |
| Thiolated chitosan | 80.00 |
| Hyprolose | 45.00 |
| Mannitol | 10.50 |
| Colloidal silicon dioxide | 1.50 |
| Sodium stearyl fumarate | 3.00 |
| Tablet weight | 180.00 |

TABLE 4

| Ingredient | mg/tablet |
|---|---|
| Glatiramer acetate | 40.00 |
| Thiolated carbomer | 80.00 |
| Hyprolose | 25.00 |
| Mannitol | 30.50 |
| Colloidal silicon dioxide | 1.50 |
| Sodium stearyl fumarate | 3.00 |
| Tablet weight | 180.00 |

All excipients, excluding the Sodium stearyl fumarate, were sieved (mesh size 800 μm) and mixed together with a lyophilized powder of glatiramer acetate in a tumble blender for 15 min. After adding the lubricant through a sieve (mesh size 500 μm) the mixing was continued for further 3 min in a tumble blender. The powder mixture was compressed on an excentric press to 9 mm round biconvex tablets with a hardness of 70-100 Newtons.

Alternatively, tablets are prepared as round flat tablets with a hardness of 60-150 Newtons.

### Example 2

### Tablet Formulations

Oral tablets containing 20 mg glatiramer acetate per tablet are prepared with the compositions set for the in Tables 5-8.

TABLE 5

| Ingredient | mg/tablet |
|---|---|
| Glatiramer acetate | 20.00 |
| Carbomer (Carbopol ® 974 P) | 50.00 |
| Hyprolose | 15.00 |
| Mannitol | 12.00 |
| Colloidal silicon dioxide | 1.00 |
| Sodium stearyl fumarate | 2.00 |
| Tablet weight | 100.00 |

TABLE 6

| Ingredient | mg/tablet |
|---|---|
| Glatiramer acetate | 20.00 |
| Chitosan | 45.00 |
| Hyprolose | 30.00 |
| Mannitol | 2.00 |
| Colloidal silicon dioxide | 1.00 |
| Sodium stearyl fumarate | 2.00 |
| Tablet weight | 100.00 |

TABLE 7

| Ingredient | mg/tablet |
|---|---|
| Glatiramer acetate | 20.00 |
| Thiolated chitosan | 45.00 |
| Hyprolose | 30.00 |
| Mannitol | 2.00 |
| Colloidal silicon dioxide | 1.00 |
| Sodium stearyl fumarate | 2.00 |
| Tablet weight | 100.00 |

TABLE 8

| Ingredient | mg/tablet |
|---|---|
| Glatiramer acetate | 20.00 |
| Thiolated carbomer | 50.00 |
| Hyprolose | 15.00 |
| Mannitol | 12.00 |
| Colloidal silicon dioxide | 1.00 |
| Sodium stearyl fumarate | 2.00 |
| Tablet weight | 100.00 |

Preparation of Tablets

All excipients, excluding the lubricant, were sieved (mesh size 800 μm) and mixed together with a lyophilized powder of glatiramer acetate in a tumble blender for 15 min. After adding the lubricant through a sieve (mesh size 500 μm) the mixing was continued for further 3 min in a tumble blender. The powder mixture was compressed on an excentric press to 7 mm round biconvex tablets with a hardness of 70-100 Newtons.

Alternatively, tablets are prepared as round flat tablets with a hardness of 60-150 Newtons.

### Example 3

Oral tablets are prepared according to Examples 1-2, above or Example 7, below. A batch of oral tablets is stored at room temperature (about 25° C.) and under refrigeration (about 4° C.). Samples from each batch are periodically examined for stability of the glatiramer acetate. The results demonstrate that glatiramer acetate stability in the oral tablets of the present invention is acceptable.

### Example 4

Oral tablets are prepared according to Examples 1-2, above or Example 7, below. Oral tablets are placed on one side of a sample of porcine buccal tissue in a Franz cell according to Example 1, above. Media from the acceptor compartment on the other side of the buccal tissue is sampled and permeability of glatiramer acetate is assessed. The results demonstrate permeation of glatiramer acetate across a sample of buccal tissue.

### Example 5

Oral tablets are prepared according to Examples 1-2, above or Example 7, below. Oral tablets are placed in apparatus 1 or apparatus 2 according to USP and dissolution of the drug is measured. After 3 hours the tablet is completely eroded. The results demonstrate that release of glatiramer acetate stability from the oral tablets of the present invention is acceptable.

### Second Series of Experiments

### Example 6

Buccal tablets were prepared with the formulation shown in Table 8.

TABLE 8

| Compound | mg/tablet | %, by weight |
|---|---|---|
| Galtiramer acetate | 50.00 | 33.33 |
| Carbopol 974P | 40.00 | 26.67 |
| Klucel HF | 10.00 | 6.67 |
| Parteck M 200 | 47.00 | 31.33 |
| Aerosil 200 | 1.00 | 0.67 |
| Pruv | 2.00 | 1.33 |

Tablets were produced under dry conditions, e.g. direct compression or dry granulation/compaction. Tablets were prepared as small, flat, round disks approximately 8 mm in diameter and approximately 2.6 mm thick.

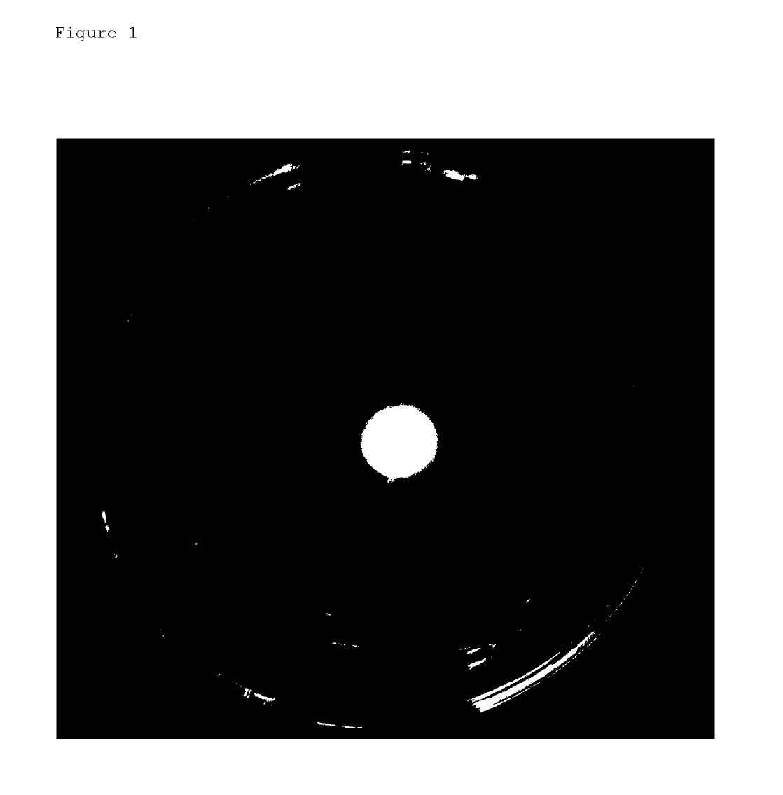

A tablet was placed on glass under a small amount of water in order to simulate the conditions present in the buccal pouch. The tablet was observed to swell but stay solid; it did not fall apart. Over the course of two hours the thickness of the tablet remained, the diameter of the tablet was observed to shrink and a cloudy ring appeared around the tablet as it slowly eroded. Results are shown in FIG. 1.

### Example 7

### Transport and Preparation of the Skin

Porcine buccal tissue was obtained from a slaughterhouse. Immediately after slaughter of the pig, pieces bearing the buccal tissue were dissected from the cheek and stored in PBS pH 7.4 and cooled on ice. The buccal tissue was isolated from the inner cheek with a scalpel and used fresh. Subsequently, the suitability of the tissue biopsy was assessed. The exclusion criteria were tissue damage or scarring.

Freshly prepared buccal tissue was cut into stripes. Tissue sections with a thickness of approx. 700-800 μm were then prepared. The dermatome was applied to the buccal tissue surface and the tissue was cut with 24 mm punch.

Permeation Study

The cylindrical Franz cell is a diffusion chamber comprising an upper and a lower part between which the porcine buccal tissue was clamped. The two halves of the cell were held together by means of a ball and socket clamp. The lower (acceptor) chamber has a volume of approx. 12 ml, while the volume of the upper (donor) chamber is variable. The tissue specimens are punched out immediately prior to insertion in the Franz cells. The tissue is always inserted with the connective tissue (lamina propia and submucosa) facing downwards so that the mucosal epithelium layer is uppermost.

The medium temperature was adjusted to 37° C. and continuously stirred at a rate of 400 rpm. The diffusion area of the porcine buccal tissue in the Franz cell was approx. 1.77 cm². Experiments were performed with six replicates for glatiramer acetate containing buccal tablets.

Buccal tablets were prepared as in Example 7 and were placed in the donor chamber of the Franz cells and the PBS buffer. The tablet was rinsed with the applied buffer in the Franz cell two or three times to ensure complete moisturizing of the tablet.

Experiments utilizing the GA solution were performed in triplicate. For each replicate, 300 μl of the formulation was applied per 1.77 cm² porcine buccal tissue at the start of the experiment. For experiments with permeation enhancer 100 μl DMSO was applied to the buccal tissue 30 minutes before the glatiramer acetate solution was applied because glatiramer acetate is not soluble in a mixture of DMSO/PBS 50:50 v/v %.

Permeation through the porcine buccal tissue into the acceptor medium was monitored over a period of 4 hours. The acceptor medium was sampled at 6 different points of time (30, 60, 90, 120, 180 and 240 min).

Determination of Glatiramer Acetate

Table 9 shows results for the permeation studies described above.

TABLE 9

| Sample | sampling time [h] | Total permeation in μg/cm² |
|---|---|---|
| | | average of n = 6 cells |
| Buccal Tablets | 0.5 | 34.61 |
| | 1 | 92.82 |
| | 1.5 | 136.53 |
| | 2 | 179.09 |
| | 3 | 253.47 |
| | 4 | 297.33 |
| | | average of n = 3 cells |
| Glatiramer | 0.5 | 153.4 |
| acetate | 1 | 156.3 |
| solution | 1.5 | 185.9 |
| without DMSO | 2 | 265.3 |
| incubation | 3 | 268.8 |
| | 4 | 329.6 |
| Glatiramer | 0.5 | 165.8 |
| acetate | 1 | 211.5 |
| solution with | 1.5 | 255.0 |
| DMSO | 2 | 277.9 |
| incubation | 3 | 324.6 |
| | 4 | 264.7 |

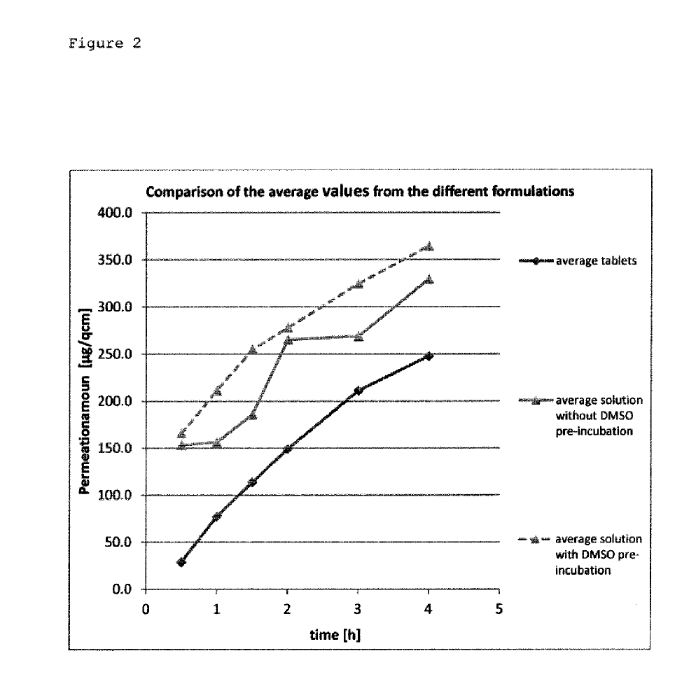

The results are shown in FIG. 2 which displays the average permeation of the different formulations. Tablets (diamond markers), glatiramer acetate solution without pre-incubated tissue (square markers, solid line) and glatiramer acetate solution with DMSO pre-incubated tissue (square markers, dotted line).

### DISCUSSION

Copaxone® is formulated as prefilled syringes containing 1 mL solution with 20 mg of glatiramer acetate. However, in the preparation of buccal tablet formulations of glatiramer acetate, applicants have surprisingly discovered that glatiramer acetate becomes sticky and forms clots which interfere with formulation of the buccal tablets. After much experimentation, applicants have determined that production of the tablets under dry conditions, e.g. using direct compression or dry granulation/compaction, provides an effective solution to this problem and results in buccal tablets which effectively deliver glatiramer acetate across the buccal membrane and have favorable dissolution properties. See, Example 8 and FIG. 1, above.

1. An oral tablet comprising glatiramer acetate in an amount from about 10 percent to about 60 percent by weight and one or more gel forming agents in a total amount up to about 90 percent by weight.

2. The oral tablet of claim 1, wherein the glatiramer acetate is present in an amount from about 10 percent to about 40 percent by weight; in an amount from about 15 percent to about 50 percent by weight; in an amount from about 25 percent to about 40 percent by weight; or in an amount from about 30 percent to about 35 percent by weight.

3-5. (canceled)

6. The oral tablet of claim 1, wherein the one or more gel forming agents are present in a total amount from about 20 percent to about 90 percent by weight; in a total amount from about 20 percent to about 40 percent by weight; in a total amount from about 30 percent to about 35 percent by weight.

7-8. (canceled)

9. The oral tablet of claim 1, further comprising a filler present in an amount up to about 80 percent by weight; in an amount up to about 60 percent by weight; in an amount up to about 50 percent by weight; in an amount up to about 40 percent by weight; in an amount from about 25 percent to about 40 percent by weight; or in an amount from about 30 percent to about 35 percent by weight.

10-14. (canceled)

15. The oral tablet of claim 1, further comprising a glidant present in an amount up to about 3 percent by weight; in an amount from about 0.3 percent to about 1.0 percent by weight; in an amount from about 0.5 percent to about 1.5 percent by weight; or in an amount from about 0.6 percent to about 0.8 percent by weight.

16-18. (canceled)

19. The oral tablet of claim 1, further comprising a lubricant present in an amount up to about 10 percent by weight; in an amount up to about 5 percent by weight; in an amount from about 1 percent to about 3 percent by weight; or in an amount from about 1.0 percent to about 1.5 percent by weight.

20-22. (canceled)

23. The oral tablet of claim 1, wherein the one or more gel forming agents are selected from the group consisting of carbomer, carbomer (sodium salt), hydroxypropylcellulose, chitosan, thiolated chitosan, thiolated carbomer, ethylcellulose, gelatine, hydroxyethylcellulose, methylcellulose, carboxymethylcellulose, sodium carboxymethlcellulose, gummi arabicum, xanthan gum, carboxymethyl cellulose and carrageen; or selected from the group consisting of carbomer, hydroxypropylcellulose, chitosan, thiolated chitosan, thiolated carbomer, ethylcellulose, gelatine, hydroxyethylcellulose, methylcellulose, carboxymethylcellulose, gummi arabicum, xanthan gum, carboxymethyl cellulose and carrageen.

24. (canceled)

25. The oral tablet of claim 9, wherein the filler is selected from the group consisting of mannitol, lactose, saccharose, sucrose, dextrose, isomalt, sorbitol, calcium phosphate, calcium carbonate, calcium silicate, magnesium carbonate, magnesium oxide, glucopyranosyl mannitol and calcium sulfate.

26. The oral tablet of claim 15, wherein the glidant is selected from the group consisting of silicon dioxide, colloidal silicon dioxide, a fumed silica, a hydrophobic fumed silica, magnesium aluminometasilicate and magnesium stearate.

27. The oral tablet of claim 19, wherein the lubricant is selected from the group consisting of sodium stearyl fumerate, a stearate, talcum powder, a fatty acid, glycerol dibehenate, hexadecanoic acid, polyethylene glycol (PEG) and magnesium stearate.

28. The oral tablet of claim 1, wherein
a) the one or more gel forming agents comprises carbomer, wherein the carbomer is present in an amount from about 10 percent to about 60 percent by weight, in an amount from about 20 percent to about 30 percent by weight, or in an amount from about 25 percent to about 30 percent by weight;
b) the one or more gel forming agents comprises hydroxypropylcellulose, wherein the hydroxypropylcellulose is present in an amount from about 1 percent to about 40 percent by weight, in an amount from about 2 percent to about 10 percent by weight, in an amount from about 6 percent to about 8 percent by weight, or in an amount of about 7 percent by weight; or
c) the tablet has a hardness of about 60 Newtons to about 150 Newtons, of about 60 Newtons to about 110 Newtons, of about 70 Newtons to about 120 Newtons, of about 70 Newtons to about 100 Newtons, or of about 80 Newtons to about 90 Newtons.

29-39. (canceled)

40. The oral tablet of claim 1, wherein the tablet is a round flat tablet.

41. The oral tablet of claim 1, wherein the tablet has a diameter from about 6 mm to about 10 mm; or a diameter of about 8 mm.

42. (canceled)

43. The oral tablet of claim 41, wherein the tablet has a thickness of about 2.6 mm.

44. The oral tablet of claim 1, wherein the tablet contains from about 10 mg glatiramer acetate to about 100 mg glatiramer acetate; from about 40 mg glatiramer acetate to about 60 mg glatiramer acetate; or about 50 mg glatiramer acetate.

45-46. (canceled)

47. The oral tablet of claim 1, comprising
(a) about 40 mg glatiramer acetate, about 80 mg carbomer, about 25 mg hydroxypropylcellulose, about 30.5 mg mannitol, about 1.5 mg colloidal silicon dioxide and about 3 mg sodium stearyl fumerate;
(b) about 20 mg glatiramer acetate, about 50 mg carbomer, about 15 mg hydroxypropylcellulose, about 12 mg mannitol, about 1 mg colloidal silicon dioxide and about 2 mg sodium stearyl fumerate;
(c) about 40 mg glatiramer acetate, about 80 mg chitosan, about 45 mg hydroxypropylcellulose, about 10.5 mg mannitol, about 1.5 mg colloidal silicon dioxide and about 3 mg sodium stearyl fumerate;
(d) about 20 mg glatiramer acetate, about 45 mg chitosan, about 30 mg hydroxypropylcellulose, about 2 mg mannitol, about 1 mg colloidal silicon dioxide and about 2 mg sodium stearyl fumerate;
(e) about 40 mg glatiramer acetate, about 80 mg thiolated chitosan, about 45 mg hydroxypropylcellulose, about 10.5 mg mannitol, about 1.5 mg colloidal silicon dioxide and about 3 mg sodium stearyl fumerate;
(f) about 20 mg glatiramer acetate, about 45 mg thiolated chitosan, about 30 mg hydroxypropylcellulose, about 2 mg mannitol, about 1 mg colloidal silicon dioxide and about 2 mg sodium stearyl fumerate;
(g) about 40 mg glatiramer acetate, about 80 mg thiolated carbomer, about 25 mg hydroxypropylcellulose, about 30.5 mg mannitol, about 1.5 mg colloidal silicon dioxide and about 3 mg sodium stearyl fumerate;
(h) about 20 mg glatiramer acetate, about 50 mg thiolated carbomer, about 15 mg hydroxypropylcellulose, about 12 mg mannitol, about 1 mg colloidal silicon dioxide and about 2 mg sodium stearyl fumerate; or
(i) about 50 mg glatiramer acetate, about 40 mg carbomer, about 10 mg hydroxypropyl cellulose, about 47 mg mannitol, about 1 mg fumed silica and about 2 mg sodium stearyl fumerate.

48-55. (canceled)

56. A method of delivering glatiramer acetate across a buccal membrane comprising orally administering an oral tablet of claim 1.

57. The method of claim 56, wherein the tablet is placed between the cheek and gum; is placed sublingually; or is placed in the mouth at bedtime.

58-59. (canceled)

60. A process of making a pharmaceutical composition containing glatiramer acetate, comprising the steps of:
a) mixing the glatiramer acetate with one of more excipients under dry conditions; and
b) forming the pharmaceutical composition.

61. The process of claim 60, wherein the pharmaceutical composition is a buccal tablet, or wherein step b) is performed by either dry granulation or direct compression.

62-63. (canceled)
